(19) United States
(12) Patent Application Publication
(10) Pub. No.: US 2023/0233462 A1
(43) Pub. Date: Jul. 27, 2023

(54) TOPICAL SOLUTION OF EFINACONAZOLE WITH HIGH SUBUNGUAL PENETRATION

(71) Applicant: THE MATERIA COMPANY LIMITED, San Gwann, SGN (MT)

(72) Inventor: Matthaios VIDALIS, San Gwann (MT)

(21) Appl. No.: 18/007,957

(22) PCT Filed: Jun. 3, 2021
(86) PCT No.: PCT/EP2021/064946

§ 371 Date: Dec. 2, 2022

(30) Foreign Application Priority Data
Jun. 3, 2020 (MT) 4428

(51) Int. Cl. A61K 9/08; A61K 31/454; A61K 47/22; A61K 47/24; A61K 47/10; A61K 47/18; A61K 47/12

(52) U.S. Cl. CPC A61K 9/08; A61K 31/454; A61K 47/22; A61K 47/24; A61K 47/10; A61K 47/18; A61K 47/12

(57) ABSTRACT

A new topical solution with high subungual penetration for the treatment of a fungal infection comprising efinaconazole, a volatile polar organic solvent, at least one phenolic antioxidant and at least one phospholipid.

### FIELD OF THE INVENTION

The present invention relates to a new pharmaceutical topical solution with high subungual penetration for the treatment of a fungal infection.

### BACKGROUND OF THE INVENTION

Efinaconazole is a well-known pharmaceutical active ingredient used for the topical treatment of onychomycosis of the toenail(s) due to Trichophyton rubrum and Trichophyton mentagrophytes. Efinaconazole is prone to chemical degradation by oxidation and is hydrophobic due to its nature.

Topical treatment is preferred, because the adverse events and drug interactions of systemic antifungal agents is avoided. Efinaconazole 10% solution is marketed under the brand name Jublia® for the treatment of onychomycosis. Treatment of onychomycosis with topical treatments is challenging because infection is embedded within the nail and is difficult to reach. The nail component is hard keratin. The keratin of nails has been classified as “hard trichocyte keratins”. In the dorsal and intermediate layers, the nail contains significant amount of phospholipids.

The penetration rates of antifungal drugs with known topical formulations through the nail plate is low, especially in long-standing disease (see Rich, J Drugs Dermatol. 2015; 14 (1), 58-62).

Formulations useful for the topical delivery of efinaconazole in the treatment of onychomycosis and other triazole antifungal drugs have been described in, e.g., U.S. Pat. Nos. 8,486,978 and 10,342,875. Said formulations comprise siloxanes which are suspected endocrine disruptors and reproductive toxins.

In view of the above state of art, the present inventors have come up with a stable pharmaceutical topical solution comprising efinaconazole and phospholipids which provides similar effect than other known topical solutions without the use of siloxanes. Further, the novel mode of delivery achieves better penetration of the pharmaceutical solution through the nail plate.

### SUMMARY OF THE INVENTION

The present invention relates to a new pharmaceutical topical solution with high subungual penetration for the treatment of a fungal infection. In a first embodiment, the new pharmaceutical topical solution comprises efinaconazole, at least one phospholipid and a volatile polar organic solvent.

In some embodiments, the amount of efinaconazole is about 10% (w/w). In some embodiments, the amount of efinaconazole is in the range of about 8% (w/w) to about 12% (w/w).

In some embodiments, the at least one phospholipid is phosphatidylcholine.

In some embodiments, the amount of phospholipid is 0.01-10%. In some embodiments pharmaceutical topical solution comprises 0.01-10% phosphatidylcholine.

In some embodiments, the volatile polar organic solvent is selected from ethyl acetate, 1-propanol, isopropanol, acetone and mixtures thereof. In some embodiments, the volatile polar organic solvent is ethyl acetate. In some embodiments pharmaceutical topical solution comprises 45-85% of ethyl acetate, 1-propanol, isopropanol, acetone and mixtures thereof. In some embodiments pharmaceutical topical solution comprises 45-85% ethyl acetate.

In some embodiments, the topical solution further comprises a phenolic antioxidant. In some embodiments the topical solution comprises 0.01%-5% phenolic antioxidant.

In some embodiments, the topical solution further comprises at least one chelating agent.

In some embodiments, the topical solution further comprises a co-solvent.

In some embodiments, the topical solution is substantially free of siloxanes.

Embodiment 1. A pharmaceutical topical solution comprising efinaconazole, a volatile polar organic solvent, at least one phenolic antioxidant and at least one phospholipid.

Embodiment 2. A pharmaceutical topical solution according to embodiment 1 comprising
a. efinaconazole,
b. 45-85% volatile polar organic solvent
c. 0.01-5% phenolic antioxidant
d. at least one chelating agent
e. 0.01-10% of at least one phospholipid
f. optionally, a co-solvent

Embodiment 3. A pharmaceutical topical solution according to embodiments 1 to 2 wherein the phospholipid is at least one phosphatidylcholine.

Embodiment 4. A pharmaceutical topical solution according to any one of embodiments 1 to 2 wherein the volatile polar organic solvent selected from the group ethyl acetate, 1-propanol, isopropanol and acetone.

Embodiment 5. A pharmaceutical topical solution according to embodiments 2 wherein the phenolic antioxidant is butylated hydroxyanisole (BHA), butylated hydroxytoluene (BHT), β, γ, δ-tocopherol, dl-α-tocopherol, 2,6-di-tert-butyl-4-ethylphenol, 2,4-di-tert-butylphenol, 2,6-di-tert-butylphenol or mixtures thereof.

Embodiment 6. A pharmaceutical topical solution according to embodiments 2 to 5 wherein the chelating agent is selected from ethylenediamine, a-ketoglutaric acid and mixtures thereof.

Embodiment 7. A pharmaceutical topical solution according to embodiments 1 to 6 wherein the volatile polar organic solvent is ethyl acetate

Embodiment 8. A pharmaceutical topical solution according to any one of embodiments wherein the efinaconazole, is about 10%.

Embodiment 9. A process for preparing the pharmaceutical topical solution of any of the preceding embodiments comprising:
i) dissolving efinaconazole, at least one phenolic antioxidant, at least one phospholipid and optionally at least one chelating agent in a first chamber comprising the volatile polar organic solvent.
ii) optionally, dissolving at least one chelating agent in the co-solvent in different chamber
mix solutions until homogenized

Embodiment 10. A method of treating a fungal infection comprising administering a therapeutically effective amount of a formulation according to any preceding embodiments to a patient in need of such treatment.

Embodiment 11. The method of embodiment 10, wherein the fungal infection is onychomycosis.

### BRIEF DESCRIPTION OF THE FIGURES

FIG. 1 shows results from in vitro penetration study of the Reference Product Jublia®, Example 1, Example 2, Example 3 and Example 4 in inert medium-like-putty (Play Doe).

### DETAILED DESCRIPTION OF THE INVENTION

The present invention relates to a new pharmaceutical topical solution with high subungual penetration for the treatment of a fungal infection. The pharmaceutical topical solution comprises the pharmaceutical agent efinaconazole and at least one phospholipid contained within the delivery system to allow passage to the nail.

The term “efinaconazole” in the context of the present invention, refers to (2R ,3R)-2-(2,4-difluorophenyl)-3-(4-methylenepiperidine-1-yl)-1-(1H-1,2,4-triazole-1-yl)butane-2-ol and pharmaceutically acceptable salts thereof.

The term “phospholipid” in the context of the present invention, suitable for use in the present invention include phosphatidylethanolamine (PE) and phosphatidylserine (PS), phosphatidylcholine (PC), lecithin, or mixtures thereof. The phospholipid can be isolated from natural sources (for example, egg yolk, soybean or other oily seed including safflower, sunflower and olive, and brain tissue) or can be produced synthetically. In a preferred embodiment, phosphatidylcholine is used. In either case, known techniques can be used for purification of the phospholipids (see, for example, J. of American Oil Chemists Soc. 42:53-56 (1965)).

The term “phosphatidylcholine” in the context of the present invention, is a class of phospholipids linked to choline. The compounds are a major component of cell membranes and can are obtainable from egg yolk, ox liver, marine animals, krill oil or soybeans. In practice, it showed that the origin of phosphatidylcholines influences their biological and chemical effects considerably. According to the invention the phosphatidylcholine (PC) can be selected from the group comprising 1-palmitoyl-2 oleoyl-sn-glycero-3-phosphocholine (POPC), natural (non-hydrogenated) or hydrogenated soy bean PC, natural or hydrogenated egg PC, dipalmitoyl phosphatidylcholine (DPPC), dimyristoyl phosphatidylcholine (DMPC) or 1,2 dioleyl-SN-glycero-3-phosphocholine (DOPC), 1-oleoyl-palmitoyl phosphatidylcholine (OPPC), diasterroyl phosphatidylcholine (DSPC), monostearoylphosphatidylcholine (MSPC), diarachidoylphosphatidylcholine (DAPC) and mixtures thereof.

The term “chelate” in the context of the present invention, is a chemical compound composed of a metal ion and a chelating agent. A chelating agent is a substance whose molecules can form several bonds to a single metal ion. A chelating agent is a multidentate ligand. An example of a simple chelating agent is ethylenediamine and a-ketoglutaric acid. Preferably ethylenediamine or a-ketoglutaric acid or mixtures thereof are used in some embodiments. In some embodiments the at least one chelating agent is a mixture of ethylenediamine and α-keto-glutaric acid in a ratio of 1:10 to 10:1. The term “antioxidants” in the context of the present invention, is compounds which scavenge free radicals by donating hydrogen to them, and they produce relatively stable antioxidant radicals with low standard reduction potential. Antioxidants slow down the oxidation rates by a combination of scavenging free radicals, chelating pro-oxidative metals, quenching singlet oxygen and photosensitizers. The higher stability of antioxidant radicals than the radicals in the finished product, is due to resonance delocalization throughout the phenolic ring structure. Preferably the bond dissociation energy for O—H of phenolic antioxidants is less than 80 kcal/mol. In a preferred embodiment the phenolic antioxidants are tocopherols, butylated hydroxytoluene (BHT), butylated hydroxyanisole (BHA), 2,6-di-tert-butyl-4-ethylphenol, 2,4-di-tert-butylphenol, 2,6-di-tert-butylphenol, tert-butylhydroquinone (TBHQ), propyl gallate (PG), lignans, flavonoids, and phenolic acids, ubiquinone (coenzyme Q), carotenoids, ascorbic acid, amino acids and mixtures thereof. In a further preferred embodiment butylated hydroxytoluene (BHT), β, γ, δ-tocopherol, dl-α-tocopherol, 2,6-di-tert-butyl-4-ethylphenol, 2,4-di-tert-butylphenol, 2,6-di-tert-butylphenol and mixtures thereof are used.

As indicated above, compositions suitable for use in the method to which the invention relates include at least one phospholipid, preferably at a concentration of 0.1 to about 10.0% (w/v).

Polar solvents decrease the radical scavenging activity of the antioxidants due to the intermolecular hydrogen bonding between oxygen or nitrogen in a polar solvent and OH group in phenolic antioxidants. Suitable pharmaceutically accepted volatile polar organic solvents are liquids such as esters, alcohols, ketones and saturated hydrocarbons with a high vapor pressure (greater than about 2 kPa at 20° C.) and boiling point less than about 100° C. at atmospheric pressure. Examples of suitable volatile polar organic solvents are ethyl acetate, butyl acetate, methyl acetate, 1-propanol, isopropanol (isopropyl alcohol), ethanol, acetone, methyl ethyl ketone and methyl isobutyl ketone. In a preferred embodiment the pharmaceutically accepted volatile polar organic solvents is chosen from ethyl acetate, 1-propanol, isopropanol, acetone and mixtures thereof. In a preferred embodiment the pharmaceutically accepted volatile polar organic solvents is ethyl acetate.

Co-solvents such as Glycerol, Polyethylene Glycols (PEGs), Propylene Glycol, Polypropylene Glycols, (PPGs), water and mixtures thereof, can be used. In a preferred embodiment Polypropylene Glycols (PPGs), water and mixtures thereof can be used.

In certain embodiments, the composition of the invention is free of volatile siloxanes. Siloxanes, also known as silicones, are cyclic and/or linear saturated cyclic and liner silicone-oxygen hydrides. Examples of cyclic siloxanes include polydimethylcyclosiloxanes, generally known as cyclomethicones (such as cyclopentasiloxane, cyclotetrasiloxane, decylmethylcyclopentasiloxane, and the like). Examples of linear siloxanes include linear polysiloxanes (such as hexamethyldisiloxane, octamethyltrisiloxane, and the like).
a. In one embodiment the pharmaceutical topical solution comprises efinaconazole, 45-85% volatile polar organic solvent
b. 0.01-5% phenolic antioxidant
c. at least one chelating agent
d. 0.01-10% of at least one phospholipid
e. optionally, a co-solvent

In another embodiment the pharmaceutical topical solution comprises efinaconazole,
a. 45-85% volatile polar organic solvent, from the group ethyl acetate, 1-propanol, isopropanol and acetone
b. 0.01-5% phenolic antioxidant
c. at least one chelating agent
d. 0.01-10% of at least one phosphatidylcholine
e. optionally, a co-solvent selected from the group glycerol, polyethylene glycols (PEGs), propylene glycol, polypropylene glycols (PPGs), water or mixtures thereof.

In another embodiment the pharmaceutical topical solution comprises efinaconazole,
a. 45-85% volatile polar organic solvent, from the group ethyl acetate, 1-propanol, isopropanol and acetone
b. 0.01-5% phenolic antioxidant
c. at least one chelating agent selected from the group ethylenediamine, a-ketoglutaric acid and mixtures thereof
d. 0.01-10% of at least one phosphatidylcholine
e. optionally, a co-solvent selected from the group glycerol, polyethylene glycols (PEGs), propylene glycol, polypropylene glycols (PPGs), water or mixtures thereof.

The term “reference product” refers to a currently or previously marketed efinaconazole solution, also described as the “originator” or “branded product” serving as a comparator in the studies. An “originator” or “branded” product are examples of a reference product. The preferred “reference product” is Jublia® solution marketed in USA.

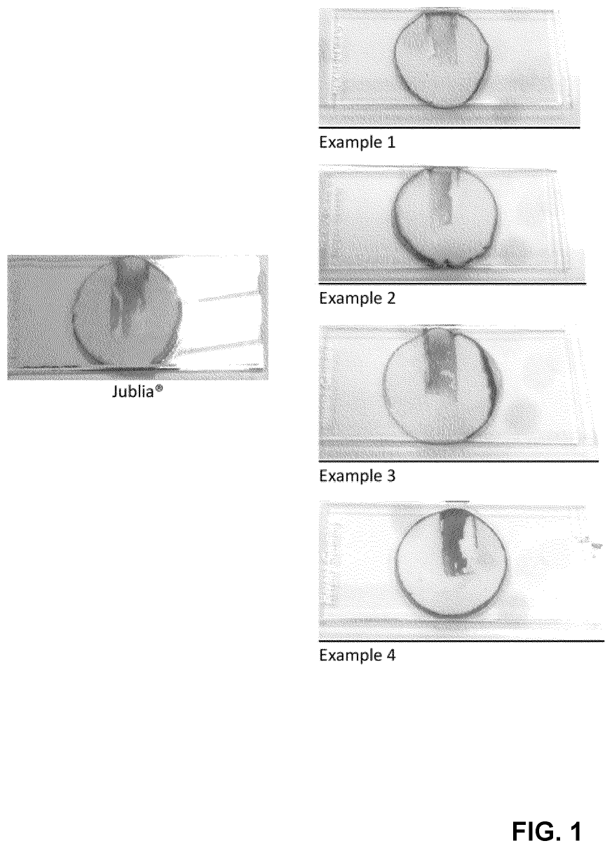

An in vitro penetration study, based on known studies (see Bhatt & Pillai, J. Pharm. Sci., 2015; 104 (7), 2177-2182) was used by the inventors to study the subungual penetration of the embodiments of the invention compared with the reference product. A pliable, non-absorbing, and inert medium-like-putty (Play Doh®) was used. A set amount of Play Doh® was rounded and placed against a glass slide. The use of a glass slide and Play Doh® aims to minimize the variability associated with human nail tissue and offer an experimental tool to standardize assessments of spreading efficiency. A thin aluminum foil of standard thickness was used to create an artificial uniform depression to mimic the air gap that often exists in onychomycotic nails. A second glass slide was placed on top, sandwiching the putty. The aluminum foil was then carefully removed. Two drops of each composition dyed red with red food coloring to aid visibility, were placed at the tip of the slide and observed. The results are demonstrated in FIG. 1. In all embodiments, the gap is seen to be fully filled indicating that the vehicle formulation penetrates efficiently.

### EXAMPLES

The pharmaceutical compositions as described herein may be illustrated by the following examples which are not to be construed as limiting the scope of the invention: The “reference product” is Jublia® 10% Efinaconazole solution marketed in USA. Examples 1-4, formulated as shown in Table 1, contained 10% efinaconazole by weight, various antioxidants and phosphatidylcholine. Examples 1-4 were prepared according to the general manufacturing process:

Step a: In the appropriate amount of ethyl acetate, dissolve efinaconazole, phosphatidylcholine, polypropylene glycol 2000 and δ-tocopherol (Example 1) or dl-α-tocopherol (Example 2) or Butylated Hydroxyanisole (Example 3) or 2,6-di-tert-butyl-4-ethylphenol and/or 2,4-di-tert-butylphenol and/or 2,6-di-tert-butylphenol (Example 4).

Step b: In another vessel, dissolve ethylenediamine and α-ketoglutaric acid in the appropriate amount of water.

Step c: Mix both solutions obtained by step a and step b until a clear, yellowish solution forms.

Alternatively, and if water is not used, dissolve ethylenediamine and α-ketoglutaric acid in the solution of step a. Skip step c.

TABLE 1
Pharmaceutical topical composition of Example 1, 2, 3 and 4

| | Example 1 | Example 2 | Example 3 | Example 4 |
|---|---|---|---|---|
| Ingredients | (w/w %) | (w/w %) | (w/w %) | (w/w %) |
| Efinaconazole | 10 | 10 | 10 | 10 |
| Ethyl Acetate | 45-85 | 45-85 | 45-85 | 45-85 |
| δ-tocopherol | 0.10-5.00 | — | — | — |
| (δ-Vitamin E) | | | | |
| dl-α-tocopherol | — | 0.10-5.00 | — | — |
| (Vitamin E) | | | | |
| Butylated | — | — | 0.10-2.00 | — |
| Hydroxyanisole | | | | |
| (BHA) | | | | |
| 2,6-di-tert-butyl-4- | — | — | — | 0.10-2.00 |
| ethylphenol | | | | |
| and/or | | | | |
| 2,4-di-tert- | | | | |
| butylphenol | | | | |
| and/or | | | | |
| 2,6-di-tert- | | | | |
| butylphenol | | | | |
| Phosphatidylcholine | 0.10-10.00 | 0.10-10.00 | 0.10-10.00 | 0.10-10.00 |
| (PC - Phospholipon | | | | |
| 90 G) | | | | |
| Polypropylene Glycol | 5.00-30.00 | 5.00-30.00 | 5.00-30.00 | 5.00-30.00 |
| Mw 2000 (PPG 2000) | | | | |
| Ethylenediamine | 0.00001-0.01 | 0.00001-0.01 | 0.00001-0.01 | 0.00001-0.01 |
| (EDA) | | | | |
| α-ketoglutaric acid | 0.001-0.10 | 0.001-0.10 | 0.001-0.10 | 0.001-0.10 |
| Water, purified | 0-5 | 0-5 | 0-5 | 0-5 |

Comparative stability results are summarized in Table 2. The results for tested products Reference product, Example 1, Example 2, Example 3 and Example 4 after 0, 3 and 6 months in accelerated conditions (40° C.±2° C., 75%±5% RH) are presented in the table below: Impurity results after 0, 3 and 6 months in accelerated conditions (40° C.±2° C., 75%±5% RH) Total impurities are acceptable since they are all below the ICH qualification thresholds. The Reference product used in the comparative study is Jublia® marketed in USA.

TABLE 2
Comparative stability results

| Storage | Reference | | | | |
|---|---|---|---|---|---|
| Conditions | product | Example 1 | Example 2 | Example 3 | Example 4 |
| Accelerated | Total | Total | Total | Total | Total |
| Conditions | Impurities | Impurities | Impurities | Impurities | Impurities |
| at 40° C./ | | | | | |
| 75% RH | | | | | |
| t = 0 | 0.00% | 0.00% | 0.00% | 0.00% | 0.00% |
| 3 Months | 0.16% | 0.04% | 0.14% | 0.00% | 0.04% |
| 6 Months | 0.23% | 0.03% | 0.33% | 0.06% | 0.09% |

### Stability Tests Methods

Impurity tests were determined by HPLC method in which column: Restek Roc C18 (150 mm×4.6 mm, 5 μm); injection volume: 25 μL; wavelength: UV, 260 nm; column temperature: 30° C.

1. A pharmaceutical topical solution comprising efinaconazole, a volatile polar organic solvent, at least one phenolic antioxidant and at least one phospholipid.

2. A pharmaceutical topical solution according to claim 1 comprising
a. efinaconazole,
b. 45-85% volatile polar organic solvent
c. 0.01-5% phenolic antioxidant
d. at least one chelating agent
e. 0.01-10% of at least one phospholipid
f. optionally, a co-solvent

3. A pharmaceutical topical solution according to claims 1 to 2 wherein the phospholipid is at least one phosphatidylcholine.

4. A pharmaceutical topical solution according to any one of claims 1 to 2 wherein the volatile polar organic solvent selected from the group ethyl acetate, 1-propanol, isopropanol and acetone.

5. A pharmaceutical topical solution according to claim 2 wherein the phenolic antioxidant is butylated hydroxyanisole (BHA), butylated hydroxytoluene (BHT), β, γ, δ-tocopherol, dl-α-tocopherol, 2,6-di-tert-butyl-4-ethylphenol, 2,4-di-tert-butylphenol, 2,6-di-tert-butylphenol or mixtures thereof.

6. A pharmaceutical topical solution according to claims 2 to 5 wherein the chelating agent is selected from ethylenediamine, a-ketoglutaric acid and mixtures thereof.

7. A pharmaceutical topical solution according to claims 1 to 6 wherein the volatile polar organic solvent is ethyl acetate

8. A pharmaceutical topical solution according to any one of claims 1 to 7 wherein the efinaconazole, is about 10%.

9. A process for preparing the pharmaceutical topical solution of any of the preceding claims comprising:
i) dissolving efinaconazole, at least one phenolic antioxidant, at least one phospholipid and optionally at least one chelating agent in a first chamber comprising the volatile polar organic solvent.
ii) optionally, dissolving at least one chelating agent in the co-solvent in different chamber
mix solutions until homogenized

10. A method of treating a fungal infection comprising administering a therapeutically effective amount of a formulation according to any preceding claims to a patient in need of such treatment.

11. The method of claim 10, wherein the fungal infection is onychomycosis.
